(19) United States
(12) Patent Application Publication
(10) Pub. No.: US 2007/0282214 A1
(43) Pub. Date: Dec. 6, 2007

(54) TECHNIQUE TO CHARACTERIZE PROXIMAL AND PERIPHERAL NITRIC OXIDE EXCHANGE USING CONSTANT FLOW EXHALATIONS AND AN AXIAL DIFFUSION MODEL

(75) Inventors: Steven C. George, Irvine, CA (US); Peter Condorelli, Irvine, CA (US); Hye-Won Shin, Irvine, CA (US)

Correspondence Address: MYERS DAWES ANDRAS & SHERMAN, LLP, 19900 MACARTHUR BLVD.,, SUITE 1150, IRVINE, CA, 92612, US

(73) Assignee: The Regents of the University of California, Oakland, CA (US)

(21) Appl. No.: 11/748,895

(22) Filed: May 15, 2007

Related U.S. Application Data
(60) Provisional application No. 60/747,377, filed on May 16, 2006.

(51) Int. Cl. A61B 5/08

(52) U.S. Cl. 600/532; 128/204.22

(57) ABSTRACT

An apparatus and method to characterize NO gas exchange dynamics in human lungs, including performing a plurality of breathing maneuvers of substantially constant flow rates within a predetermined range, measuring data relating to at least one of an NO concentration and an NO elimination rate as a function of exhaled volume or exhalation flow rate, applying a lung model to the measured data, and obtaining at least one parameter indicative of disease states of the lung based on the lung model and the measured data, wherein the lung model, when applied in the predetermined range, predicts a substantially linear relationship between the NO elimination rate and the exhalation flow rate.

### CROSS-REFERENCE TO RELATED APPLICATIONS

The present application claims priority pursuant to 35 USC 119 of U.S. Provisional Application Ser. No. 60/747,377, filed May 16, 2006, the disclosure of which is herein incorporated by reference in its entirety.

### GOVERNMENT RIGHTS

This invention was made with Government support under Grant No. HL070645, awarded by the National Institutes of Health. The Government has certain rights in this invention.

### BACKGROUND OF THE INVENTION

1. Field of the Invention

The invention relates generally to apparatus and method for characterizing airway nitric oxide to characterize disease states of lungs.

2. Description of the Prior Art

Nitric oxide (NO) was first detected in the exhaled breath of healthy and asthmatic humans in the 1990s. The exhaled NO arises from both airway and alveolar regions of the lungs, which provides an opportunity to characterize region-specific inflammation. Since NO modulates many functions in the lungs (e.g., smooth muscle tone, neurotransmission, and inflammation), there has been considerable interest in understanding NO as a potentially useful noninvasive biological marker.

Early work established a strong inverse relationship between the NO concentration and the exhalation flow, yet a positive relationship between the elimination rate (product of concentration and flow) and exhalation flow. To explain these observations, two-compartment models (2CM) were developed in which both the airways (rigid tubes) and the alveolar (flexible balloon) regions were sources of exhaled NO. The 2CM was attractive because the analytical solution could easily be adapted to create algorithms that analyzed breathing maneuvers with mathematical techniques in which exhaled NO could be partitioned into alveolar (peripheral) and airway (proximal) contributions. This led to the rapid application of these techniques to characterize proximal and peripheral NO in a range of normal and pathological conditions including exercise, asthma, COPD, cystic fibrosis, and scleroderma.

The simplicity of the 2CM is both its strength and weakness. While the initial description of the model considered the increasing cross-sectional area with distance into the airway tree (i.e., the “trumpet” shape), the subsequent early descriptions neglected this feature, and all of the early models neglected axial (as opposed to radial) diffusion of NO in the gas phase.

Therefore, what is needed is: 1) a technique that is simple to perform and is capable of determining both airway and alveolar NO concentrations; and 2) which includes more realistic models.

### BRIEF SUMMARY OF THE INVENTION

In one aspect, embodiments of the invention provide a technique that employs realistic models, yet achieve simplified data analysis by utilizing a linear regime in measured data.

An illustrated embodiment is a method to characterize nitric oxide (NO) gas exchange dynamics in a lung, comprising (1) performing a plurality of breathing maneuvers of substantially constant flow rates within a predetermined range; (2) measuring data relating to at least one of an NO concentration and an NO elimination rate as a function of exhaled volume or exhalation flow rate; (3) applying a lung model to the measured data; and (4) obtaining at least one parameter indicative of disease states of the lung based on the lung model and the measured data, wherein the lung model, when applied in the predetermined range, predicts a substantially linear relationship between the NO elimination rate and the exhalation flow rate.

The method further comprises characterizing proximal (airway) and peripheral (alveolar) airway NO using the lung model that includes axial diffusion of NO and a trumpet shape of the airways.

In one embodiment, characterizing proximal (airway) and peripheral (alveolar) airway NO comprises using a logarithmic description of a cross sectional area of the airways.

In one embodiment, characterizing proximal (airway) and peripheral (alveolar) airway NO comprises applying Fick's 1^{st} law of steady-state diffusion.

In one embodiment, characterizing proximal (airway) and peripheral (alveolar) airway NO further comprises assuming a constant flux of NO from the airways.

In one embodiment, performing the plurality of breathing maneuvers comprises exhaling over the predetermined range of flow rates of 50-500 ml/s. In one embodiment, performing the plurality of breathing maneuvers comprises exhaling over a preferred range of flow rates of 100-250 ml/s.

In one embodiment, performing the plurality of breathing maneuvers comprises performing a series of constant-flow single exhalation (vital capacity) breathing maneuvers. In one embodiment, the method further comprises inhaling NO-free air to total lung capacity and immediately exhaling against a flow restrictor to maintain a constant flow rate in the range of 50-500 ml/s. In one embodiment, the method further comprises measuring the concentration of NO in the exhaled breath as a function of exhalation flow rate. In one embodiment, the method further comprises measuring the concentration of NO in the exhaled breath at a series of different exhalation flow rates. In an embodiment, the amount of NO in the exhaled breath depends on the exhalation, the amount of NO coming from the alveolar region and from the airway region, the method further comprising determining peripheral and proximal NO exchange in the lung by applying the model to the measured NO concentration, the model including a trumpet shape of the airway tree and axial diffusion of NO.

In one embodiment, determining peripheral and proximal NO exchange in the lung is used to track inflammatory diseases of the airways such as asthma or diseases of the alveolar region such as pneumonia. In another embodiment, determining peripheral and proximal NO exchange in the lung is used to track asthma and is used to follow the efficacy of treatment, diagnose asthma, or predict onset of an acute exacerbation.

In one embodiment, the method further comprises obtaining a relationship between a measured elimination rate of NO versus a measured exhalation flow. In one embodiment, the method further comprises applying a linear least squares fitting to the relationship between the elimination rate of NO versus the measured exhalation flow. In one embodiment, the method further comprises obtaining an alveolar concentration of NO and a maximum airway flux of NO from the linear fit.

In one embodiment, the method further comprises plotting a relationship between a measured NO concentration and a measured exhaled volume. In one embodiment, the method further comprises obtaining a plateau NO concentration from the relationship between the measured NO concentration and the exhaled volume. In one embodiment, the exhaled volume is between 5-10 exhaled airway volumes.

In one embodiment, the method further comprising partitioning a proximal and a peripheral NO exchange in the lung.

According to another aspect of the invention, an apparatus is provided for characterizing nitric oxide (NO) gas exchange dynamics in a lung to diagnose a disease state of the lung, comprising: (1) means for performing a plurality of breathing maneuvers of substantially constant flow rates within a predetermined range; means for measuring data relating to NO concentration as a function of exhaled volume or exhalation flow rate; (3) means for obtaining a linear relationship from the measured data based on a realistic lung model, the linear relationship reducing data analysis loads; and (4) means for characterizing a proximal (airway) and a peripheral (alveolar) airway NO. In one embodiment, the plurality of breathing maneuvers are limited to a predetermined range of flow rates of 50-500 ml/s.

In one embodiment, the apparatus further comprises means for applying a model of the lung that includes axial diffusion of NO and the trumpet shape of the airways. In one embodiment, the apparatus further comprises means for diagnosing lung disease states based on the measured NO concentration and the realistic lung model.

According to yet another aspect, data acquisition and analysis may be included in a commercially available software package.

A computer readable medium in accordance with embodiments of the invention containing instructions, the instructions comprising:

(1) obtaining data relating to NO concentration as a function of exhaled volume or exhalation flow rate over a predetermined range resulting from a plurality of substantially constant-flow breathing maneuvers; (2) applying a lung model to the data; and (3) obtaining at least one parameter indicative of disease states of the lung from a linear relationship among the data based on the lung model.

While the apparatus and method has or will be described for the sake of grammatical fluidity with functional explanations, it is to be expressly understood that the claims, unless expressly formulated under 35 USC 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 USC 112 are to be accorded full statutory equivalents under 35 USC 112. The invention can be better visualized by turning now to the following drawings wherein like elements are referenced by like numerals.

### BRIEF DESCRIPTION OF THE DRAWINGS

FIG. 1 is a block diagram of the apparatus in which the method of the illustrated embodiment is practiced.

FIG. 2 is a schematic of the airway trumpet model.

FIGS. 3A-3C are graphs illustrating the determination of plateau exhaled concentrations.

FIG. 4 is a graph demonstrating the relationship between \dot{V}_{NO} and \dot{V} for eight subjects, including the best fit line.

FIG. 5 is a graph showing the determined parameters characterizing peripheral (CA_{NO}) and proximal (J′aw_{NO}).

FIG. 6 is a graph showing the dependence of the function f on exhalation flow over the flow range 100<\dot{V}<250 ml/s. f is a function of the exhalation flow, the molecular diffusivity of NO in the insufflating gas (i.e., D_{NO,air} or axial diffusion), and the cross-sectional area of the airway tree at the airway-alveolar junction (i.e., the shape of the trumpet),

FIG. 7 is a flowchart showing steps of performing a method in accordance with an embodiment of the invention.

The invention and its various embodiments can now be better understood by turning to the following detailed description of the preferred embodiments which are presented as illustrated examples of the invention defined in the claims. It is expressly understood that the invention as defined by the claims may be broader than the illustrated embodiments described below.

### DETAILED DESCRIPTION OF THE PREFERRED EMBODIMENTS

Although several techniques have been presented that employ a breath hold (single or multiple) or tidal breathing maneuver, the most common method by far is a series of single exhalation maneuvers from maximal inspiration in which the exhalation flow is held constant during a single exhalation, but different exhalation flows are used (i.e., multiple constant flow exhalations). In addition, the constant flow exhalation is the recommended maneuver of both the American Thoracic Society (ATS) and the European Respiratory Society (ERS), usually performed at a single exhalation flow.

Although some of Applicant's work has incorporated axial diffusion and the trumpet-shape of the airway tree into the governing material balance equations of the 2CM, the solutions have required cumbersome numerical techniques or considered a transient no flow (breath hold) condition, which may be difficult for some patients. Yet such trumpet geometry and gas-phase axial diffusion of NO are critical features of NO exchange that should be considered in the analytical methods.

The goals of the current study are three-fold: 1) develop a steady state model of NO exchange which considers axial diffusion and the trumpet shape of the airway tree; 2) use the model to develop an algorithm which analyzes a series of steady state constant flow exhalations and partitions exhaled NO into proximal and peripheral components; and 3) compare the performance of the new model with the earlier simpler model that neglects axial diffusion and the trumpet shape of the airway tree.

A technique to estimate key flow-independent parameters characteristic of NO exchange in the lungs has been described in U.S. Pat. No. 6,866,637. A tidal breathing technique has been described in U.S. Patent Application Pub. No. 2003/0229290. Another technique has been described in U.S. patent application Ser. No. 11/636,027, filed Dec. 8, 2006. These patents and patent applications are assigned to the assignee of the present invention, and the disclosures of which are incorporated herein by reference in their entirety.

In the previously described methods: 1) a single 20-second breath hold was used and combined with the entire exhalation phase including a prescribed decreasing exhalation flow as described in U.S. Pat. No. 6,866,637; 2) a series of tidal breathing NO concentration traces was analyzed to determine airway and alveolar exchange; or 3) a protocol used multiple different breath holds of different time, and examined the exhaled profile that included air from the airways, and did not have any prescribed exhalation flow rate.

All techniques employ a least squares fitting routine between the exhalation profile and a model to determine NO exchange parameters. Up to this point, a simple technique that employs commonly used single exhalation constant flow maneuvers with a model that considers the trumpet shape of the airways with axial diffusion of NO has not been developed.

In the past, the technique which utilizes constant flow maneuvers considers a model that neglects the trumpet shape of the airway tree, which is modeled as if it were a cylinder, and neglects axial diffusion. More recent breath hold technique, as disclosed in U.S. patent application Ser. No. 11/636,027, considers the trumpet shape of the airway tree and axial diffusion, but relies on the subject holding his or her breath, and does not characterize the alveolar region. Incorporating the trumpet shape of the airway tree and axial diffusion complicates the governing mathematical equations, such that new simplifying assumptions were employed in the illustrated embodiment of the present invention to overcome this disadvantage.

A method and apparatus is disclosed herein to characterize proximal (airway) and peripheral (alveolar) airway NO exchange in the lung using a mathematical model of the lungs that considers several important new features, namely axial diffusion of NO and the trumpet shape of the airways, a method of analysis of experimental data, and a series of experimental breathing maneuvers characterized by a constant flow single exhalations (vital capacity) over a range of flows 50-500 ml/s.

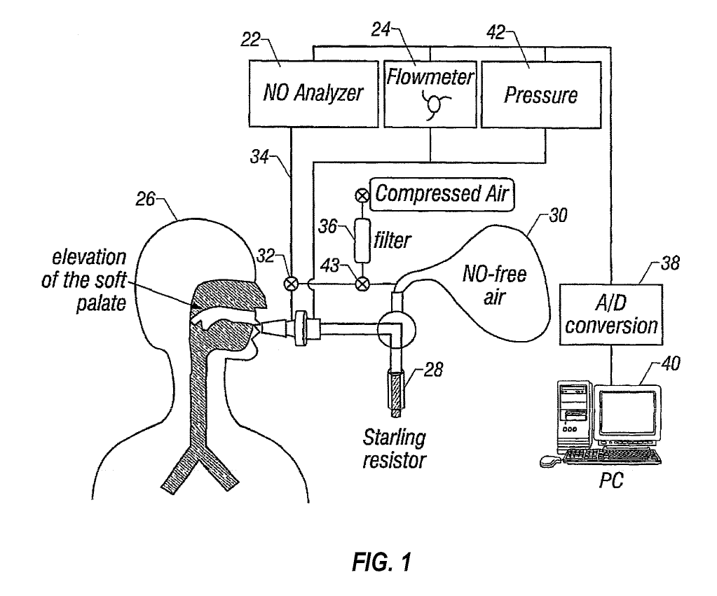

A schematic of the experimental apparatus is shown in FIG. 1, which is a block diagram of the experimental setup used to collect the exhalation profiles. The identical apparatus, used differently, is disclosed in U.S. Pat. No. 6,866,637 and in U.S. patent application Ser. No. 11/636,027.

A subject 26 inhales NO-free air to total lung capacity, and immediately begins to exhale against a flow restrictor, e.g., the starling resistor 28, which helps maintain the flow constant in a predetermined range, e.g., 50-500 ml/s. The concentration of NO in the exhaled breath is measured as a function of exhaled volume. The maneuvers are repeated at a series of different exhalation flows. The amount of NO in the exhaled breath depends on the exhalation, the amount of NO coming from the alveolar region, and the amount of NO from the airway region.

By applying a model to the data taking into account the trumpet shape of the airway tree and axial diffusion of NO, peripheral and proximal NO exchange in the lungs can be determined. The primary advantage of the illustrated embodiments of the invention is a more physiological and physical, and thus more accurate, description of NO exchange dynamics in the lungs. The result is a more accurate estimate of NO exchange in the lungs while maintaining mathematical simplicity.

This information can then be used to track inflammatory diseases of the airways such as asthma, or diseases in the alveolar region such as pneumonia. The illustrated embodiments of the invention can be used to monitor the inflammatory state of the airway tissue or alveolar tissue in the lungs. In the case of asthma, the technique may be used to follow the efficacy of treatment, diagnose asthma, or predict the onset of an acute exacerbation.

The flow, pressure, and NO analog signals are captured by the analytical instruments and converted to a digital signal using an A/D converter 38. The digital data is stored on a computer 40 for further analysis. A software containing instructions to perform measurements, data analysis, and diagnostic suggestions, may be installed in the computer, e.g., stored in computer readable media such as memory, hard drive, floppy disk, compact disk, or flash memory.

While computer 40 is arranged and configured by conventional programming to perform the functions disclosed in this specification, it is to be expressly understood that computer 40 may be substituted by equivalent means, such as logic circuits, digital signal processors and other analog and/or digital signal processing circuitry.

In the present study, exhaled NO concentration and exhalation flow are measured from healthy non-smoking non-asthmatic adults with no history of respiratory diseases. The protocol focused on an exhalation flow range that is practical and easy to perform in terms of the magnitude of the flow itself, as well as the number of flows and breathing maneuvers. The flow rates are sufficiently high to ensure that the wall flux of NO from the airway tree (J_{aw}_{_{NO}}, pl/s) is constant (independent of flow) and approaches the maximum airway wall flux of NO (J′_{aw}_{_{NO}}, pl/s). This condition is safely met for flows greater than 100 ml/s in healthy adults, and greatly simplifies the solution of the governing equations as discussed below.

Thus, the target flows were 100, 150, 200, and 250 ml/s. These flows have been commonly employed by other research groups, and can be performed by nearly all subjects. Furthermore, by performing each maneuver in triplicate, the number of exhalation flows is limited to twelve for each subject. While the predetermined range of flow rate and the target flow rates are given above for exemplary purposes, it is to be expressly understood that the range of the target flow rates may be different from those disclosed herein.

The group consisted of 8 subjects (5 female) with (mean±SD) age, height, weight, and FEV₁ (Vmax229; Sensormedics, Yorba Linda, Calif.), of 31±5 years, 165±10.1 cm, 62.3±12.3 kg, and 3.5±0.6 l (102±4.5% predicted), respectively (see Table 1 for details). Each exhalation flow was achieved using flow restrictors. Flow, pressure, and NO concentration (model 280B, Ionics, Inc. Boulder, Colo.) for each maneuver were recorded simultaneously. The protocol was approved by the Institutional Review Board at the University of California, Irvine, and written informed consent was obtained from each subject.

It has been previously demonstrated that the slope of the exhalation NO profile in phase III is statistically negative (between 4-12% of the concentration per liter exhaled) at a constant exhalation flow. Thus, when determining CE_{NO} for multiple exhalation flows, it is important to analyze the exhaled concentration over a similar lung volume. It is also desirable to ensure that the airway tree had been sufficiently emptied of the inspired air, and that the variation in lung volume between subjects is considered.

Thus, exhaled concentration as a function of the number of airway volumes (V_{aw}) exhaled is plotted. V_{aw} was estimated as the sum of the subjects age in years and the ideal body weight in pounds. Then, CE_{NO} was calculated as the mean concentration between five and ten exhaled airway volumes. The minimum of five airway volumes was chosen to completely washout the airway tree of the inspired air and allow a steady flow to be achieved. A maximum of ten was chosen to assure analysis over a time window (approximately 3-7 seconds depending on the flow) that is similar to the recommended guidelines of the American Thoracic Society (3 seconds) and those of the European Respiratory Society. A breathing maneuver was excluded if the standard deviation of the exhalation flow over this same exhaled volume range exceeded 5% (i.e., the maneuver was not considered “constant exhalation flow”).

Models for vital capacity maneuvers at constant exhalation flow assume that the concentration and flux of NO in the proximal (airway) and peripheral (alveolar) regions of the human lungs approach steady state (i.e., independent of time), and a steady (“plateau”) concentration (CE_{NO}) is achieved in the exhaled air at the mouth. The Trumpet Model with Axial Diffusion (TMAD) characterizes airway geometry by appropriately scaling the lengths and diameters of Weibel's data (Weibel E., Morphometry of the human lung. Berlin: Springler-Verlag, 1963.) of the human airway tree, based on the conducting airway volume (V_{aw}) of the oral cavity, oropharynx, and generations 0-17 for each subject (see Table 1).

TABLE 1
Physical Characteristics of Subjects.

| | | Age | Hgt | Wgt | Iwgt | V_{aw} | FEV₁ |
|---|---|---|---|---|---|---|---|
| | Gen | (yrs) | (cm) | (kg) | (kg) | (ml) | (l, % predicted) |
| 1 | F | 35 | 150 | 44 | 50 | 145 | 2.59, 104 |
| 2 | M | 29 | 168 | 66 | 65 | 173 | 3.59, 96 |
| 3 | M | 31 | 170 | 73 | 67 | 179 | 3.62, 93 |
| 4 | F | 25 | 160 | 49 | 57 | 150 | 3.23, 106 |
| 5 | M | 27 | 178 | 79 | 73 | 188 | 4.75, 103 |
| 6 | F | 28 | 178 | 69 | 67 | 175 | 3.63, 102 |
| 7 | F | 30 | 163 | 53 | 58 | 158 | 3.17, 105 |
| 8 | F | 39 | 157 | 66 | 55 | 160 | 3.07, 107 |
| Mean | | 31 | 165 | 62 | 62 | 166 | 3.50, 102 |
| SD | | 5 | 10 | 12 | 8 | 15 | 0.6, 4.5 |

Gen: gender;
Hgt: height;
Wgt: body weight;
Iwgt: ideal body weight;
V_{aw}: volume of the airway compartment estimated in ml as the sum of the subjects ideal body weight (lbs) plus age (yrs) (34);
FEV₁: forced expiratory volumes in one sec (liters and % predicted).

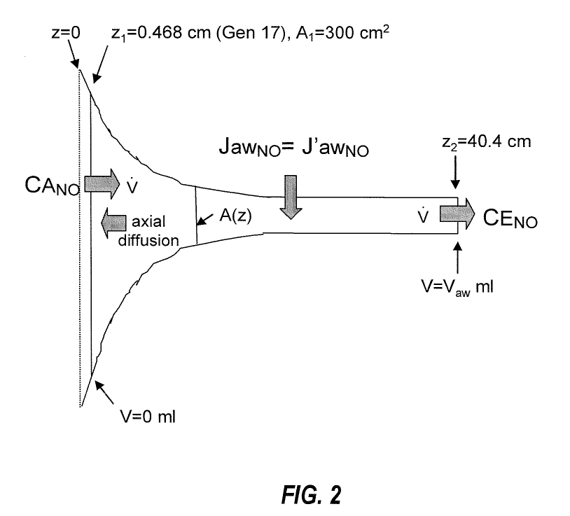

FIG. 2 shows the mapping of airway dimensions to a “trumpet geometry”; using the following logarithmic relationship:

$$A = {{A}_{1} ( \frac{z}{{z}_{1}} )}^{- m} \qquad ( 1 )$$

where A is the airway cross sectional area, z is the axial position, subscript “2” refers to the axial position at the mouth (subscript “1” refers to the axial position at the end of generation 17), and m=2 provides an excellent match to the data of Weibel.

A solution to the steady-state diffusion equation generating the following solution for the exhaled concentration of NO at the mouth can be obtained as discussed later in the text,

$${C}_{ENO} = {C}_{ANO} + \frac{{J}_{awNO}^{′}}{\dot{V}} \cdot f ( \dot{V} , {𝒟}_{NO , air ,} {A}_{1} ) \qquad ( 2 )$$

where f is a function of the exhalation flow, the molecular diffusivity of NO in the insufflating gas (i.e., [symbol] _{NO,air} or axial diffusion), and the cross-sectional area of the airway tree at the airway-alveolar junction (i.e., the shape of the trumpet).

Alveolar air with NO concentration CA_{NO} exits the alveolar region at position z₁ (generation #17) and is transported towards the mouth (position z₂) by convection at a steady flow \dot{V}. NO is added to the air stream at rate Jaw_{NO} (pl/s) from the airway wall. Because the flow is larger than 100 ml/s, Jaw_{NO} can be considered constant and equal to the maximum airway flux J′aw_{NO} (9). Thus, the concentration increases with z-position and the concentration at the mouth (the exhaled concentration, CE_{NO}) is described by Eq. 2.

Axial diffusion of NO is described by Fick's 1^{st} law of diffusion and transport NO from high to low concentration; thus, NO is simultaneously transported by diffusion in the axial direction back towards the alveolar region. The airway volume is defined as the volume between positions z₁ and z₂, and is estimated in ml as the sum of the subjects age in years plus ideal body weight in pounds. The cross sectional area of the trumpet decreases with increasing z-position and is determined by the relationship in Eq. 1 by mapping the airway dimensions to that of the Weibel symmetric bifurcating airway tree.

The key assumption in the solution to the governing equation is that the flux of NO from the airway tree is a constant (i.e., does not depend on flow), and thus the solution is valid for approximately exhalation flows >100 ml/s in healthy adults. Note that as f approaches unity, the simple solution of the 2CM is attained.

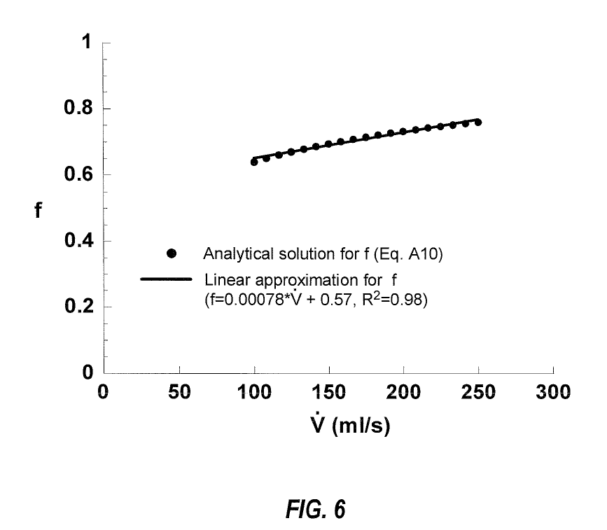

Eq. 2 can be simplified further by limiting the flow to the range to 100<\dot{V}<250 ml/s, which is the range commonly employed in experimental studies including the present study. In this range, f is nearly a linear function of \dot{V} (R²=0.98, see FIG. 6) and can be approximated by f=(0.00078 s/ml)*\dot{V}+0.57. If this relationship is inserted into Eq. 2, and both sides of the equation are multiplied by \dot{V}, the following linear relationship for the elimination rate (\dot{V}_{NO}, pl/s) of NO as a function of flow is attained,

$${\dot{V}}_{NO} = ( {C}_{ANO} + {J}_{awNO}^{′} \cdot 0.00078 ) \dot{V} + \frac{{J}_{awNO}^{′}}{1.7} \qquad ( 3 )$$

where the factor 1.7 is the inverse of 0.57.

Thus, the model predicts that a plot of \dot{V}_{NO} versus \dot{V} produces a linear relationship in which the slope, S, is equal to CA_{NO}+J′_{aw}_{_{NO}}*0.00078 and the intercept, I, is equal to J′_{aw}_{_{NO}}/1.7. Hence, CA_{NO} and J′_{aw}_{_{NO}} can be estimated from a plot of \dot{V}_{NO} versus \dot{V} using the TMAD and the following simple relationships,

$$\begin{matrix}TMAD ( 100 < \dot{V} < 250 ml \text{/} s ) , & \\ {C}_{ANO} = S - l ( \frac{0.00078 s \text{/} ml}{0.57} ) = S - \frac{l}{740 ml / s} , & ( 4 ) \\ {J}_{awNO}^{′} = 1.7 \cdot l , & ( 5 )\end{matrix}$$

where S is the slope and I is the y-intercept using simple linear regression. This can be contrasted with the 2CM in which CA_{NO} and J′_{aw}_{_{NO}} can be approximated as simply S and I, respectively. Values for J′_{aw}_{_{NO}} and CA_{NO} were thus determined by applying linear least squares to a plot of \dot{V}_{NO} versus \dot{V} for each subject using both the 2CM and the TMAD (Eqs. 4 and 5). The slope was constrained to be greater than or equal to zero.

It is advantageous of using \dot{V}_{NO} versus \dot{V} instead of alternate forms that utilize CE_{NO} as the dependent variable. In brief, using \dot{V}_{NO} as the dependent variable effectively places more weight on the data obtained at higher flow. This can be justified because the assumption of a constant wall flux becomes more accurate as the flow increases (and thus the model is more accurate); thus, this technique provides a more accurate estimate of J′_{aw}_{_{NO}}, and CA_{NO}.

Confidence intervals (95%) for the determined parameters were calculated assuming a normally distributed error using the t-statistic for the slope and intercept of V_{NO} versus \dot{V} for each subject. Differences between the determined parameters using the TMAD and 2CM models as well comparing the determined parameters to a mean value of zero were calculated using a paired t-test or single population t-test, respectively. Statistical significance was assumed for p<0.05.

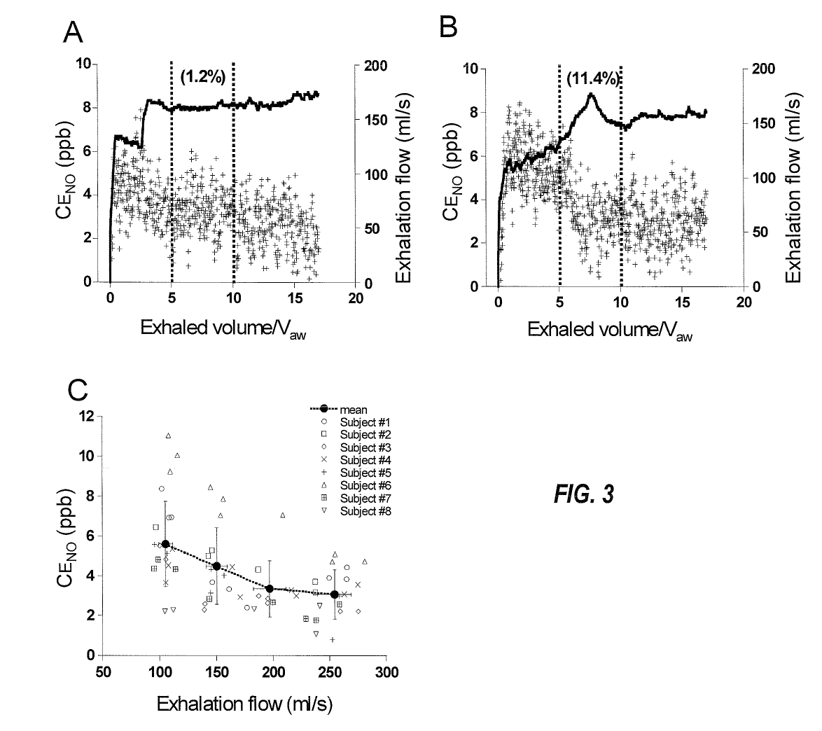

In each subject, one or more maneuvers were eliminated by not meeting the criteria for a constant exhalation flow. FIGS. 3A-3C show representative exhalation profiles from subject #1 for a typical maneuver that was included (FIG. 3A), and excluded (FIG. 3B).

More specifically, FIG. 3A shows representative exhalation flow profiles from subject #1 at the targeted exhalation flow of 150 ml/s for an exhalation maneuver included in the final analysis, and FIG. 3B shows a maneuver excluded from analysis. The “+” are NO concentration from an unfiltered signal, and the solid line is exhalation flow. The space between the vertical dashed lines represents the window of analysis (between five and ten exhaled airway volumes).

Note that in maneuver marked for inclusion, several airway volumes are needed to be exhaled before a steady flow is achieved; then, during the analysis window, the standard deviation of the flow is 1.2% (number in parenthesis). Note also that despite a constant flow, a negative slope in the NO concentration is evident highlighting the need to analyze the profiles over a constant exhaled volume window that is scaled to the subject's lung volume. In the maneuver marked for exclusion, the exhalation flow is not constant (standard deviation 11.4%) until after the analysis window. FIG. 3C summarizes the plateau NO concentrations from all eight subjects of the profiles marked for inclusion, including the mean value (solid circle) at each of the targeted exhalation flows (100, 150, 200, and 250 ml/s).

Of the 96 breathing maneuvers (12 maneuvers/subject×8 subjects), 65 (68%) were included for further analysis, the remaining 31 having been eliminated by exceeding the maximum variation in flow during the analysis window (standard deviation>5%). FIG. 3C demonstrates CE_{NO} of the profiles which met the inclusion criteria as a function of exhalation flow for all eight subjects. The data demonstrate the inverse relationship between CE_{NO} and \dot{V} for all eight subjects that has been previously reported.

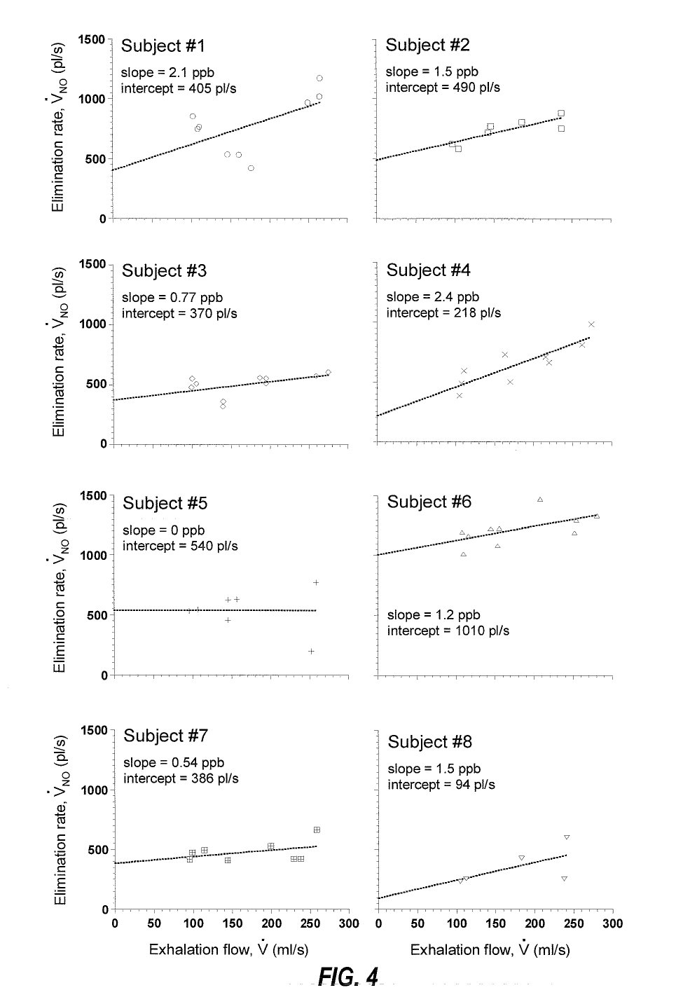

FIG. 4 demonstrates the relationship between \dot{V}_{NO} and \dot{V} for all eight subjects, including the best fit line. \dot{V}_{NO} has been calculated using the individual NO concentrations and flows shown in FIG. 3C. Note that a positive relationship between \dot{V}_{NO} and \dot{V} exists for seven out of the eight subjects. For subject #5, the best fit line (constraining the slope to be greater than or equal to zero) has a zero slope.

NO elimination rate (V_{NO}) versus exhalation flow (\dot{V}) is presented for all eight subjects using only the exhalation profiles that met the requirements for inclusion (FIG. 2). The solid line is the best fit line using linear regression and the best fit slope and intercept for this line are also shown. The slope was greater than zero for all subjects except subject #5 (equal to 0).

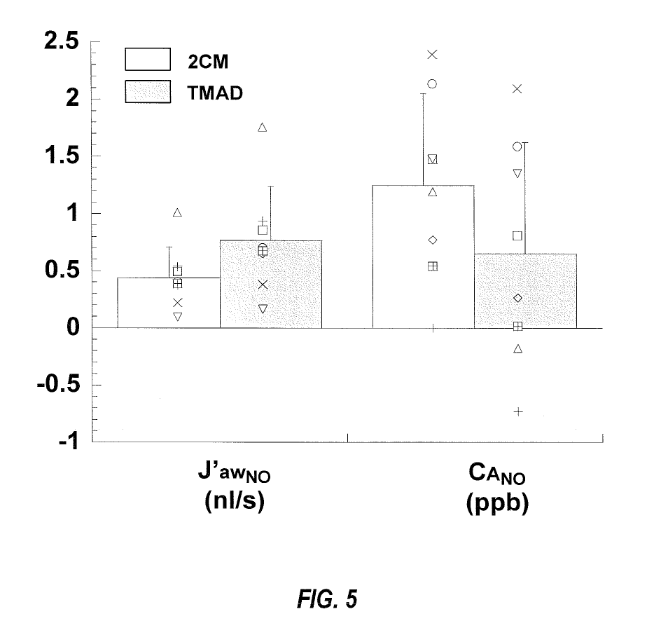

FIG. 5 compares the estimated values for CA_{NO} and J′_{aw}_{_{NO}} using the 2CM and TMAD (Eqs. 4 and 5) models. The mean (±SD) value of CA_{NO} for the TMAD model is 0.66±0.98 ppb which is not statistically different from zero (p>0.05), and statistically smaller than the mean value determined with the 2CM model (1.2±0.80 ppb, which is statistically larger than zero). The mean (±SD) value of J′_{aw}_{_{NO}} for the TMAD model is 770±470 μl/s, which is statistically larger (1.7 times, see Eq. 4) than the mean value determined from the 2CM model (440±270 pl/s).

NO exchange obtained using the 2CM and the TMAD are compared. The mean of the eight subjects (bar), standard deviation of the mean (error bar), and data points (symbols) for each of the subjects are shown. CA_{NO} and J′aw_{NO} were determined using the slope and intercept of V_{NO} vs. \dot{V} shown in FIG. 4 and Eqs. 4 and 5 in the text describing the TMAD model.

An important consideration in a method to determine unknown parameters is the uncertainty in the estimate Table 2 presents the uncertainty (95% confidence interval) in the estimates for CA_{NO} and J′_{aw}_{_{NO}} for each of the subjects and for each of the models. The 95% confidence interval for CA_{NO} spans zero for six and seven subjects, respectively, for the 2CM and TMAD models. For J′_{aw}_{_{NO}}, the 95% confidence interval spans zero for only three of the eight subjects for both models. The mean maximum deviation from the central value for J′_{aw}_{_{NO}} (for both models) is 154±225%; however, this value is significantly skewed by subject #8 in which the central value is small (94 pl/s) and the uncertainty high. If this subject is removed, the mean maximum deviation is 74±41%.

TABLE 2
Airway and Alveolar NO Exchange Parameters and Confidence Intervals.

| | 2CM | | | | | | TMAD | | | | | |
|---|---|---|---|---|---|---|---|---|---|---|---|---|
| | CA_{NO} | | | J′aw_{NO} | | | CA_{NO} | | | J′aw_{NO} | | |
| Subject | central | lower | upper | central | lower | upper | central | lower | upper | central | lower | upper |
| 1 | 2.1 | −0.56 | 4.8 | 410 | −100 | 910 | 1.6 | −1.8 | 4.8 | 710 | −170 | 1600 |
| 2 | 1.5 | 0.34 | 2.6 | 490 | 300 | 690 | 0.81 | −0.59 | 2.6 | 860 | 500 | 1200 |
| 3 | 0.77 | −0.26 | 1.8 | 370 | 190 | 560 | 0.26 | −1.0 | 1.8 | 650 | 330 | 970 |
| 4 | 2.4 | 1.1 | 3.7 | 220 | −25 | 460 | 2.1 | 0.49 | 3.7 | 380 | −40 | 800 |
| 5 | 0.00 | −3.5 | 2.8 | 540 | 40 | 1200 | −0.73 | −5.1 | 2.8 | 940 | 70 | 2000 |
| 6 | 1.2 | −0.09 | 2.5 | 1000 | 770 | 1200 | −0.18 | −1.8 | 2.5 | 1800 | 1300 | 2200 |
| 7 | 0.54 | −0.66 | 1.7 | 390 | 170 | 610 | 0.020 | −1.5 | 1.5 | 670 | 290 | 1000 |
| 8 | 1.5 | −2.0 | 5.0 | 94 | −560 | 750 | 1.40 | −3.5 | 6.1 | 160 | −980 | 1300 |
| mean | 1.2 | | | 440 | | | 0.66*^{,†} | | | 770^{†} | | |
| SD | 0.80 | | | 270 | | | 0.98 | | | 470 | | |

2CM: two-compartment model;
TMAD: trumpet model with axial diffusion;
CA_{NO}: alveolar concentration of NO;
J′aw_{NO}: maximum airway flux of NO;
“lower” and “upper” refer to the limits of the 95% confidence interval for the central determined value.
*not statistically different from zero (p > 0.05);
^{†}different from the 2CM model.

Embodiments of the invention as described above provide a technique to partition proximal and peripheral NO exchange in the lungs that incorporates previously neglected, yet relevant, physical features of the airway tree and gas exchange, while maintaining mathematical and computational simplicity. By limiting the flow range to 100-250 ml/s, both the trumpet shape of the airway tree and axial diffusion of NO can be incorporated, yet the technique still produces a solution of the governing equation and computational technique that utilizes the slope and intercept of NO elimination versus flow.

The result is a 1.7-fold larger flux of NO from the airway tree, and a near zero alveolar (peripheral) NO concentration. These results are consistent with previous, yet more sophisticated numerical models, which included the trumpet shape and axial diffusion. Thus, the technique described above provides a description of NO exchange dynamics more accurate than the previously described and commonly employed 2CM for constant flow exhalations, but maintains simplicity. Thus, the method can be used to describe proximal and peripheral NO exchange in lung disease in many different situations.

It has been previously shown that adding axial diffusion of NO alone (i.e., maintaining the cylindrical geometry) does not significantly impact NO exchange. However, the combination of axial diffusion with the trumpet shape dramatically increases the loss of NO to the alveolar region. This is due to the fact that the rate of axial diffusion is proportional to the product of the concentration gradient (change in concentration with axial position) and the cross-sectional area.

The dramatic increase in the cross-sectional area in the peripheral regions of the lungs effectively reduces the resistance of NO diffusion in the axial direction, making this a significant physical force that cannot be neglected. Because the observed concentration of NO at the mouth is unchanged, the predicted flux of NO from the airway tree must increase to account for the loss of NO due to back diffusion into the alveolar region. Previous estimates using breath hold techniques has estimated this increase to be between 2-5 fold, which is consistent with our current prediction using constant flow exhalations (1.7-fold increase).

The large pool of blood in the alveolar region provides a near-infinite sink (primarily hemoglobin) to scavenge NO. Thus, any additional NO that diffuses from the airway tree towards the alveolar region is immediately bound and does not impact the steady alveolar concentration. It has been previously demonstrated that the relative impact of axial diffusion decreases as exhalation flow increases. This is due to the shift in the balance between convection (movement of NO from the bulk flow of air) of NO and diffusion (Brownian motion of NO molecules) of NO.

The rate of convective transport of NO increases in proportion to the exhalation flow, but does not impact the rate of axial diffusive transport. Thus, as flow increases, the loss of NO to the alveolar region by diffusion decreases. This phenomenon by itself can produce a positive slope in the plot of NO elimination versus exhalation flow of approximately 1 pl/s per ml/s (ppb) over a flow range of 100-250 ml/s in healthy subjects. The relative impact should depend on the flux of NO from the airway tree. The larger the airway flux, the larger the gradient of NO in the airway tree, and thus the larger the impact of axial diffusion. This trend is predicted by the model described above.

The alveolar concentration is equal to the value of slope of the NO elimination versus flow minus a term that is proportional to the airway flux (Eq. 5). For example, for our predicted mean airway flux of 770 pl/s, the impact of axial diffusion and the trumpet shape of the airway tree can produce a slope of 0.60 ppb (1/740 s/ml=J′_{aw}_{_{NO}}/(1.7*740 s/ml), Eqs. 4 and 5) that must be subtracted from the slope to reveal the true alveolar concentration. Since the mean slope of NO elimination versus flow in our subjects was only 1.2 ppb, the predicted alveolar concentration by the TMAD is near zero (0.66 ppb) and consistent with Applicant's previous predictions using more complex numerical solutions and breath hold techniques.

The result in FIG. 5 and Table 2 that CA_{NO} is negative using the TMAD model in two subjects should not be interpreted as a true negative concentration as this has no physical meaning. The 95% confidence interval presented in Table 2 for CA_{NO} is the true range of possible values (with 95% confidence), and this range includes positives values for all subjects. The fact that the 95% confidence interval includes negative values simply reflects the noise and error in the experimental measurement and mathematical model. It is important to note that the mean value for all subjects is greater than zero, albeit not statistically different from zero.

An important limitation in the present technique is the inability to characterize the airway diffusing capacity of NO, or D_{aw}_{_{NO}}. As previous studies show, in order to estimate D_{aw}_{_{NO}}, the exhalation flow must be low enough such that the concentration of NO in the airway tree, C_{NO}, can reach a sufficiently high level to decrease the airway flux. In other words, the airway flux is no longer a constant and equal to the maximum airway flux. This phenomenon occurs for exhalation flows less than approximately 50 ml/s (5*D_{aw}_{_{NO}}) in healthy subjects. However, even when such low flows are utilized, the uncertainty in determining D_{aw}_{_{NO}} remains large, and obtaining a reliable plateau exhaled concentration is difficult for many subjects due to the necessary long exhalation time. For example, if one were to examine the same exhaled volume region as in the current study (between 5-10 exhaled airway volumes), one would need to exhale for between 30-60 seconds assuming an exhalation flow of 25 ml/s and an airway volume of 166 ml (mean of the eight subjects in this study). D_{aw}_{_{NO}} may be a useful steroid-independent parameter in asthma, and we have previously shown that a series of breath hold maneuvers may be the most accurate method to characterize its magnitude.

The TMAD model has been previously used to determine the airway NO parameters J′_{aw}_{_{NO}} and D_{aw}_{_{NO}} using a series of breath hold maneuvers. In this technique, the governing equation is unsteady, and there is no convection (analysis considers only the NO accumulation during the breath hold). In these studies, the mean J′_{aw}_{_{NO}} was 4150-4350 pl/s which is 5.4-5.6 times larger than the estimate in the current study (770 pl/s). However, the impact of axial diffusion is similar in predicting a 2.6 fold increase in J′_{aw}_{_{NO}} (e.g., 4350 μl/s as compared to 1704 pl/s in previous studies). Thus, the difference in the techniques is primarily the magnitude of J′_{aw}_{_{NO}}. This difference may be due to different subject populations. There is significant variation in NO elimination amongst the healthy population and our current study included only 8 subjects.

In the present study, the estimated J′_{aw}_{_{NO}} using the 2CM (440 pl/s) is smaller than most other estimates using constant flow exhalations (range 700-1280 pl/s, (9)). In addition, the difference in the magnitude of J′_{aw}_{_{NO}} may reflect differences in the techniques. For example, the breathhold technique is a transient technique, and the estimated value of J′_{aw}_{_{NO}} is proportional to the estimated value of V_{aw}. In contrast, the present technique depends on steady state measurements of exhaled concentration and flow, and is independent of V_{aw} (with the minor exception that the window of analysis to determine CE_{NO} is based on V_{aw}). In any event, although the relative impact of axial diffusion and the trumpet shape is consistent between the techniques, caution must be exercised in comparing absolute values of the determined parameters between techniques.

The uncertainty in estimating J′_{aw}_{_{NO}} in the current technique is significant in healthy subjects. In three of the subjects, the 95% confidence interval spanned zero, suggesting not that J′_{aw}_{_{NO}} was necessarily zero (the NO is coming from somewhere), but rather that the technique could not determine a positive value with 95% confidence. The uncertainty is due to the noise in the experimental data of plotting \dot{V}_{NO} versus \dot{V} and utilizing only 5-10 data points (depending on the subject). Additional breathing maneuvers will improve the accuracy of the estimated value at the expense of additional effort on the part of the subject. Single breath techniques with a prescribed decrease in the exhalation flow during the maneuver may provide a more accurate estimate of airway and alveolar NO contributions with much fewer breathing maneuvers, but they require more sophisticated mathematical tools and have not yet been tested with axial diffusion and the trumpet shape of the airway tree.

Finally, the flow range utilized in the current study was chosen based on the relative ease at which subjects can perform them, and the need to keep the airway flux constant (i.e., J′_{aw}_{_{NO}}>>D_{aw}_{_{NO}}*C_{NO}). However, several research groups have presented constant flow exhalations using flows larger than 250 ml/s, and in healthy subjects the airway flux may be constant for flows as low as 50 ml/s. Furthermore, the applicable flow range in disease states such as asthma has not yet been determined. It appears that both the wall concentration (C_{aw}_{_{NO}}) and D_{aw}_{_{NO}} are elevated in asthma, and thus the applicable flow range may be similar to healthy subjects (i.e., a larger D_{aw}_{_{NO}} requires a larger minimum flow, but a larger wall concentration increases J′_{aw}_{_{NO}} and reduces the minimum flow). The critical feature to determine the applicability of the current model is an observed linear relationship between \dot{V}_{NO} and \dot{V}. Nonetheless, the flow range utilized will impact the approximate linear relationship for f, and subsequent relationships for CA_{NO} and J′_{aw}_{_{NO}}.

Thus, Table 3 presents the approximation for f and relationships for CA_{NO} and J′_{aw}_{_{NO}} for several additional flow ranges that might be employed using this technique. Note that as higher flows are considered in the analysis, axial diffusion becomes less important, and the slope and intercept of \dot{V}_{NO} versus \dot{V} more closely approaches CA_{NO} and J′_{aw}_{_{NO}}, respectively.

TABLE 3
Approximation for f and subsequent relationships for
CA_{NO} and J′aw_{NO}.

| | f ≅ a * V + b | | | CA_{NO} ≅ S − I/c | |
|---|---|---|---|---|---|
| Flow range | a | | | c | J′aw_{NO} ≅ I * d |
| (ml/s) | (s/ml) | b | R² | (ml/s) | d |
| 50–250 | 0.00100 | 0.53 | 0.94 | 530 | 1.9 |
| 50–500 | 0.00056 | 0.59 | 0.89 | 1100 | 1.7 |
| 100–250 | 0.00078 | 0.57 | 0.98 | 740 | 1.7 |
| 100–300 | 0.00068 | 0.59 | 0.97 | 860 | 1.7 |
| 100–400 | 0.00055 | 0.61 | 0.95 | 1100 | 1.6 |
| 100–500 | 0.00045 | 0.63 | 0.94 | 1400 | 1.6 |

S, slope of V_{NO} (pl/s) vs V (ml/s);
I, intercept of V_{NO} (pl/s) vs V (ml/s);
R², coefficient of determination for the linear approximation of the complex function f (19 data points with evenly distributed values for exhalation flow were utilized in the linear regression).

The model considers the combination of steady state flow conditions, the trumpet shape of the airway tree (increasing cross-sectional area with distance into the lungs), and axial diffusion of NO. The technique utilizes the previously described and commonly employed plot of NO elimination vs. exhalation flow, but the presence of the trumpet shape and axial diffusion produces an alternate interpretation of the resulting slope and intercept. The result is a 1.7-fold increase in the predicted flux of NO from the airway tree, and an alveolar concentration that is near zero. The technique includes the most relevant anatomical and physical features of the lungs (i.e., the trumpet shape of the airways and axial diffusion), yet maintains simplicity by considering only steady state flows that are readily performed by most adult subjects. Thus, the technique may be useful to a broad range of investigators in characterizing proximal and peripheral NO in lung pathology.

The development of the model and the derivation of related equations discussed above are not described in details. The development of the governing equation for the model begins with a differential mass balance over a thickness Δz in the airway tube. The salient features of the model are: 1) a cross-sectional area, A, that depends on z-position (trumpet shape, Eq. 1); 2) a constant airway flux per unit volume from the airway wall (radial diffusion) equal to the total maximum airway wall flux from the entire airway tree, J′aw_{NO} (pl/s) divided by the airway volume, Vaw; 3) axial diffusion (in the z-direction) of NO in the gas phase is governed by Fick's 1^{st} law of diffusion (A*D_{NO,air}*dC_{NO}/dz, pl NO/s) where D_{NO,air} is the molecular diffusivity of NO in air; 4) convection of NO in the z-direction is characterized by the bulk exhalation flow, \dot{V} (ml/s); and, 5) steady state conditions. The result is the following form of the convective-diffusion equation describing the concentration of NO, C_{NO} (ppb or pl NO/cm³), in the airway tree as a function of position,

$$\frac{{d}^{2} {C}_{NO}}{d {z}^{2}} + [ \frac{1}{A} \frac{d A}{d z} - \frac{\dot{V}}{{𝒟}_{NO , air} {A}_{cs}} ] \frac{d {C}_{NO}}{d z} + \frac{{J}_{awNO}^{′}}{{𝒟}_{NO , air} {V}_{aw}} = 0 , \qquad ( A1 )$$

with the following two boundary conditions,

$$\begin{matrix}{C}_{NO} ( z = {z}_{1} ) = {C}_{ANO} , & ( A2 ) \\ \frac{d {C}_{NO}}{d z} ( z = {z}_{2} ) = 0. & ( A3 )\end{matrix}$$

The first boundary condition (Eq. A2) simply states that the concentration of NO entering the trumpet at position z₁ (generation 17) is equal to the alveolar concentration, CA_{NO}. The second boundary condition states that convective flow is large enough near mouth (position z₂) that the concentration gradient in the z-position is negligible or approaches zero.

The values for z₁ and z₂ are determined using the data from Weibel for generations 0-23, and from Hanna and Scherer (Hanna L M and Scherer P W, Measurement of local mass transport coefficients in a cast model of the human upper respiratory tract. J Biomech Eng 108: 12-18, 1986.) for the dimensions of the oropharynx and oral cavities. Thus, z₁=0.468 cm (end of generation 17), and z₂=40.4 cm. Note then that the airway volume can be easily estimated by integrating Adz over the length of the trumpet,

$${V}_{aw} = {\int }_{{z}_{1}}^{{z}_{2}} A ( z ) d z = {A}_{1} {z}_{1} {\int }_{1}^{{x}_{2}} {x}^{- 2} d x = {A}_{1} {z}_{1} ( 1 - {x}_{2}^{- 1} ) \qquad ( A4 )$$

where x=z/z₁ (and thus x₂=z₂/z₁=84.6) and A₁ is the cross-sectional area at position z₁ (300 cm², FIG. 2). Eq. A4 produces a value for V_{aw} of 142 ml which is in close agreement with our population mean estimate of 166 ml using the sum of the subjects' ideal body weight in lbs plus age in years.

The solution to the governing equation (Eq. A1) is most readily attained by defining the following non-dimensional parameters: φ=C_{NO}/CA_{NO}, X=z/z₁, Pe₁=z₁\dot{V}/ [symbol] _{NO,air} A₁, α=J′_{awNO}z₁²/ [symbol] _{NO,ari}V_{aw}C_{ANO} where Pe₁ is the Peclet number at z-position z₁ representing the ratio of the rate of bulk convection of NO to rate of axial diffusion, and α is proportional the ratio of the rate of radial diffusion of NO to rate of axial diffusion of NO. Inserting these relationships into Eqs. A1-A3 results in the following, simpler set of equations,

φ″+[Pe₁x²+2x⁻¹]φ′+α=0, (A5)

with boundary conditions,

φ(x=1)=1, (A6)

φ′(x=x₂)=0, (A7)

where φ′ and φ″ are the first and second derivatives with respect to x. The solution to Eq. A5 (second order inhomogeneous ordinary differential equation with variable coefficients) can be solved analytically for the concentration of NO exiting the mouth (CE_{NO}, equivalent to C_{NO} at position z₂ or φ at position x₂) by using an integrating factor, and integrating by parts. The result is,

$$φ ( {x}_{2} ) = 1 + \frac{α}{{Pe}_{1}} [ {( \frac{{Pe}_{1}}{3} )}^{1 / 3} {e}^{( {Pe}_{1} / 3 )} ( Γ ( ( \frac{{Pe}_{2}}{3} ) , \frac{2}{3} ) - Γ ( ( \frac{{Pe}_{1}}{3} ) , \frac{2}{3} ) ) - {x}_{2}^{- 1} ] , \qquad ( A8 )$$

where Pe₂ is the Peclet number at position z₂ (z₂\dot{V}/ [symbol] _{NO,air}A₂), and Γ(u,n) is the lower incomplete gamma function defined by,

$$Γ ( u , n ) = {\int }_{0}^{u} {t}^{n - 1} {e}^{- t} d t . \qquad ( A9 )$$

One can rewrite Eq. A8 by re-introducing the dimensional parameters to arrive at Eq. 2 in the main body of the text where,

$$f = \{ \frac{{( \frac{{Pe}_{1}}{3} )}^{1 / 3} {e}^{( {Pe}_{1} / 3 )} [ 1.354 - Γ ( \frac{{Pe}_{1}}{3} , \frac{2}{3} ) ] - {x}_{2}^{- 1}}{1 - {x}_{2}^{- 1}} \} . \qquad ( A10 )$$

In Eq. A10, Γ(Pe₂,2/3) has been replaced by the constant value of 1.354, which is valid for exhalation flows >10 μl/s. FIG. 6 shows the dependence of f with exhalation flow over the flow range 100<\dot{V}<250 ml/s.

Function f (Eq. A10, solid points) is plotted as a function of exhalation flow, \dot{V}(ml/s), over the flow range of 100-250 ml/s. The solid line represents the linear fit of the nineteen data points (f=0.00078*\dot{V}+0.57, R²=0.98). The function f is a monotonically increasing function of \dot{V}, and is >0.95 for \dot{V}>2.5 l/s.

### Note the near-linear relationship in which f can be approximated (R²=0.98) by the much simpler form,

f=0.00078(\dot{V})+0.57, (A11)

which can then be inserted into Eq. 2 as shown in the main text, to arrive at Eqs. 3-5.

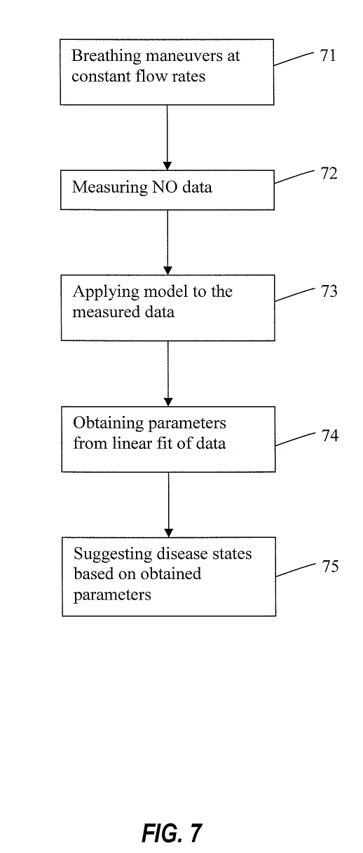

The method in accordance with embodiments of the invention is now summarized with reference to the flowchart of FIG. 7.

In step 71, a plurality of breathing maneuvers are performed, each at a different yet substantially constant flow rate. The flow rates are limited to a predetermined range as discussed earlier. The number of maneuvers need to take into account patient comfort, clinical settings, and need to provide a reasonable coverage within the predetermined range to increase the measurement accuracy. In step 72, NO data are measured. In step 73, a realistic, yet sufficiently simple model, is applied to the measured data. In step 74, parameters are obtained from a linear fit of the data. As a result of the breathing maneuvers discussed above and the simplicity of the model, a linear fit can be used thus reducing the time and computing power for data analysis. In step 75, diagnostic suggestions based on the obtained parameters may be provided to the health care providers to be used in tracking lung diseases.

Many alterations and modifications may be made by those having ordinary skill in the art without departing from the spirit and scope of the invention. Therefore, it must be understood that the illustrated embodiment has been set forth only for the purposes of example and that it should not be taken as limiting the invention as defined by the following invention and its various embodiments.

The words used in this specification to describe the invention and its, various embodiments are to be understood not only in the sense of their commonly defined meanings, but to include by special definition in this specification structure, material or acts beyond the scope of the commonly defined meanings. Thus if an element can be understood in the context of this specification as including more than one meaning, then its use in must be understood as being generic to all possible meanings supported by the specification and by the word itself.

The definitions of the words or elements of the following invention and its various embodiments are, therefore, defined in this specification to include not only the combination of elements which are literally set forth, but all equivalent structure, material or acts for performing substantially the same function in substantially the same way to obtain substantially the same result. In this sense it is therefore contemplated that an equivalent substitution of two or more elements may be made for any one of the elements in the invention and its various embodiments below or that a single element may be substituted for two or more elements in a claim.

Insubstantial changes from the claimed subject matter as viewed by a person with ordinary skill in the art, now known or later devised, are expressly contemplated as being equivalently within the scope of the invention and its various embodiments. Therefore, obvious substitutions now or later known to one with ordinary skill in the art are defined to be within the scope of the defined elements.

The invention and its various embodiments are thus to be understood to include what is specifically illustrated and described above, what is conceptionally equivalent, what can be obviously substituted and also what essentially incorporates the essential idea of the invention.

What is claimed is:

1. A method to characterize nitric oxide (NO) gas exchange dynamics in a lung, comprising:
performing a plurality of breathing maneuvers of substantially constant flow rates within a predetermined range;
measuring data relating to at least one of an NO concentration and an NO elimination rate as a function of exhaled volume or exhalation flow rate;
applying a lung model to the measured data; and
obtaining at least one parameter indicative of disease states of the lung based on the lung model and the measured data,
wherein the lung model, when applied in the predetermined range, predicts a substantially linear relationship between the NO elimination rate and the exhalation flow rate.

2. The method of claim 1, further comprising characterizing proximal (airway) and peripheral (alveolar) airway NO using the lung model that includes axial diffusion of NO and a trumpet shape of the airways.

3. The method of claim 2, wherein characterizing proximal (airway) and peripheral (alveolar) airway NO comprises using a logarithmic description of a cross sectional area of the airways.

4. The method of claim 2, wherein characterizing proximal (airway) and peripheral (alveolar) airway NO comprises applying Fick's 1^{st} law of steady-state diffusion.

5. The method of claim 4, wherein characterizing proximal (airway) and peripheral (alveolar) airway NO further comprises assuming a constant flux of NO from the airways.

6. The method of claim 1, wherein performing the plurality of breathing maneuvers comprises exhaling over the predetermined range of flow rates of 50-500 ml/s.

7. The method of claim 6, wherein performing the plurality of breathing maneuvers comprises exhaling over a preferred range of flow rates of 100-250 ml/s.

8. The method of claim 1, wherein performing the plurality of breathing maneuvers comprises performing a series of constant-flow single exhalation (vital capacity) breathing maneuvers.

9. The method of claim 8, further comprising inhaling NO-free air to total lung capacity and immediately exhaling against a flow restrictor to maintain a constant flow rate in the range of 50-500 ml/s.

10. The method of claim 9, further comprising measuring the concentration of NO in the exhaled breath as a function of exhalation flow rate.

11. The method of claim 10, further comprising measuring the concentration of NO in the exhaled breath at a series of different exhalation flow rates.

12. The method of claim 11, wherein the amount of NO in the exhaled breath depends on the exhalation, the amount of NO coming from the alveolar region and from the airway region, the method further comprising determining peripheral and proximal NO exchange in the lung by applying the model to the measured NO concentration, the model including a trumpet shape of the airway tree and axial diffusion of NO.

13. The method of claim 12, wherein determining peripheral and proximal NO exchange in the lung is used to track inflammatory diseases of the airways such as asthma or diseases of the alveolar region such as pneumonia.

14. The method of claim 12, wherein determining peripheral and proximal NO exchange in the lung is used to track asthma and is used to follow the efficacy of treatment, diagnose asthma, or predict onset of an acute exacerbation.

15. The method of claim 1, further comprising obtaining a relationship between a measured elimination rate of NO versus a measured exhalation flow.

16. The method of claim 15, further comprising applying a linear least squares fitting to the relationship between the elimination rate of NO versus the measured exhalation flow.

17. The method of claim 16, further comprising obtaining an alveolar concentration of NO and a maximum airway flux of NO from the linear fit.

18. The method of claim 1, further comprising plotting a relationship between a measured NO concentration and a measured exhaled volume.

19. The method of claim 18, further comprising obtaining a plateau NO concentration from the relationship between the measured NO concentration and the exhaled volume.

20. The method of claim 18, wherein the exhaled volume is between 5-10 exhaled airway volumes.

21. The method of claim 1, further comprising partitioning a proximal and a peripheral NO exchange in the lung.

22. An apparatus for characterizing nitric oxide (NO) gas exchange dynamics in a lung to diagnose a disease state of the lung, comprising:
means for performing a plurality of breathing maneuvers of substantially constant flow rates within a predetermined range;
means for measuring data relating to NO concentration as a function of exhaled volume or exhalation flow rate;
means for obtaining a linear relationship from the measured data based on a realistic lung model, the linear relationship reducing data analysis loads; and
means for characterizing a proximal (airway) and a peripheral (alveolar) airway NO.

23. The apparatus of claim 22, wherein the plurality of breathing maneuvers are limited to a predetermined range of flow rates of 50-500 ml/s.

24. The apparatus of claim 22, further comprising means for applying a model of the lung that includes axial diffusion of NO and the trumpet shape of the airways.

25. The method of claim 24, further comprising means for diagnosing lung disease states based on the measured NO concentration and the realistic lung model.

26. A computer readable medium containing instructions, the instructions comprising:
obtaining data relating to NO concentration as a function of exhaled volume or exhalation flow rate over a predetermined range resulting from a plurality of substantially constant-flow breathing maneuvers;
applying a lung model to the data; and
obtaining at least one parameter indicative of disease states of the lung from a linear relationship among the data based on the lung model.
